(19) United States
(12) Patent Application Publication
(10) Pub. No.: US 2023/0149017 A1
(43) Pub. Date: May 18, 2023

(54) ENDOSCOPIC SUTURING METHOD AND MEDICAL INSTRUMENT

(71) Applicant: OLYMPUS MEDICAL SYSTEMS CORP., Tokyo (JP)

(72) Inventors: Masaru YUASA, Hachioji-shi (JP); Masato TAMAI, Hachioji-shi (JP); Shogo SHINDO, Koganei-shi (JP)

(73) Assignee: OLYMPUS MEDICAL SYSTEMS CORP., Tokyo (JP)

(21) Appl. No.: 17/986,974

(22) Filed: Nov. 15, 2022

Related U.S. Application Data
(60) Provisional application No. 63/279,703, filed on Nov. 16, 2021.

(51) Int. Cl. A61B 17/10; A61B 17/00; A61B 17/08

(52) U.S. Cl. CPC A61B 17/10; A61B 17/00234; A61B 17/083; A61B 2017/0647

(57) ABSTRACT

An endoscopic suturing method includes penetrating an indwelling member into a first portion of a mucous membrane, moving the indwelling member toward a second portion of the mucous membrane while pushing the first portion of the mucous membrane and penetrating the indwelling member into the second portion of the mucous membrane with the indwelling member penetrated into the first portion of the mucous membrane, and leaving the indwelling member in a body in a state in which the indwelling member penetrates the first portion of the mucous membrane and the second portion of the mucous membrane.

### RELATED APPLICATION DATA

This application is based upon and claims the benefit of priority from U.S. Provisional Patent Application Ser. No. 63/279,703, filed on Nov. 16, 2021, the entire contents of which are incorporated herein by reference.

### FIELD OF THE DISCLOSURE

The present disclosure relates to an endoscopic suturing method and a medical instrument used in the endoscopic suturing method.

### BACKGROUND

In endoscopic treatment, medical instruments such as clip units to treat living tissue are used. These medical instruments are introduced to the treatment site by a device that can be passed through a channel of the endoscope.

Japanese Unexamined Patent Application, First Publication No. H9-38093 (Patent Document 1) describes a treatment tool used to puncture a living tissue. The treatment instrument described in Patent Document 1 can be left in the body in a state of being punctured into living tissue.

### SUMMARY

Based on the above circumstances, the object of the present disclosure is to provide an optimal medical instrument for suturing tissue and an endoscopic suturing method using the medical instrument.

The endoscopic suturing method according to the first aspect of the present invention includes penetrating an indwelling member into a first portion of a mucous membrane, moving the indwelling member toward a second portion of the mucous membrane while pushing the first portion of the mucous membrane and penetrating the indwelling member into the second portion of the mucous membrane with the indwelling member penetrated into the first portion of the mucous membrane, and leaving the indwelling member in a body in a state in which the indwelling member penetrates the first portion of the mucous membrane and the second portion of the mucous membrane.

The medical instrument of the present invention and the endoscopic suturing method using the medical instrument can suitably perform, for example, suturing of resected or defective portion.

### BRIEF DESCRIPTION OF THE DRAWINGS

FIG. 1 is a perspective view of an applicator.

FIG. 2 is a perspective view of the medical instrument according to a first embodiment.

FIG. 3 is a diagram for explaining the operation of the medical instrument.

FIG. 4 is a diagram for explaining the operation of the medical instrument.

FIG. 5 is a diagram for explaining the operation of the medical instrument.

FIG. 6 is a diagram for explaining the operation of the medical instrument.

FIG. 7 is a diagram for explaining the operation of the medical instrument.

FIG. 8 is a flow chart showing a suturing procedure using the medical instrument.

FIG. 9 is a diagram for explaining the first step of the suture treatment.

FIG. 10 is a cross-sectional view of the defective portion in the suturing direction.

FIG. 11 is a diagram for explaining the second step of the suture treatment.

FIG. 12 is a cross-sectional view of the missing portion in the second step.

FIG. 13 is a cross-sectional view of the missing portion in the second step.

FIG. 14 is a diagram for explaining the third step of the suture treatment.

FIG. 15 is a cross-sectional view of the missing portion in the third step.

FIG. 16 is a diagram for explaining the fourth step of the suture treatment.

FIG. 17 is a perspective view of a clip unit.

FIG. 18 is a flow chart showing a suturing procedure according to a second embodiment.

FIG. 19 is a flow chart showing the procedure of the fifth step of the suturing treatment.

FIG. 20 is a diagram for explaining the fifth step of the suture treatment.

FIG. 21 is a cross-sectional view of the distal end of a sheath used for suturing treatment according to a third embodiment.

FIG. 22 is a flow chart showing the procedure of the suture treatment.

FIG. 23 is a diagram for explaining the first auxiliary step of the suture treatment.

FIG. 24 is a diagram for explaining the first auxiliary step of the suture treatment.

FIG. 25 is a diagram showing a modification of the projection of the applicator.

FIG. 26 is a perspective view of a medical instrument according to a fourth embodiment.

FIG. 27 is a perspective view of a medical instrument according to another aspect of the fourth embodiment.

FIG. 28 is a perspective view of a medical instrument according to another aspect of the fourth embodiment.

FIG. 29 is a diagram for explaining treatment with the medical instrument.

FIG. 30 is a diagram for explaining treatment with the medical instrument.

FIG. 31 is a perspective view of a medical instrument according to another aspect of the same embodiment.

FIG. 32 is a perspective view of a medical instrument according to another aspect of the same embodiment.

FIG. 33 is a diagram for explaining a suturing treatment according to another aspect of the same embodiment.

FIG. 34 is a diagram for explaining a suturing treatment according to another aspect of the same embodiment.

FIG. 35 is a diagram for explaining a suturing treatment according to another aspect of the same embodiment.

FIG. 36 is a diagram showing a medical instrument according to a fifth embodiment.

FIG. 37 is a diagram showing the medical instrument.

FIG. 38 shows the medical instrument further including a stopper.

FIG. 39 shows the medical instrument further including a stopper.

FIG. 40 is a flow chart showing a suturing procedure using a medical instrument according to a sixth embodiment.

FIG. 41 is a perspective view of an applicator according to a seventh embodiment.

FIG. 42A is a side view of the hook of the applicator.

FIG. 42B is a front view of the hook viewed from direction B in FIG. 42A.

FIG. 42C is a cross-sectional view of the hook along line C-C in FIG. 42A.

FIG. 42D is a cross-sectional view of the hook along line D-D in FIG. 42A.

FIG. 43 is a perspective view of a medical instrument according to a seventh embodiment.

FIG. 44 is a diagram for explaining the operation of the medical instrument and the applicator.

FIG. 45A is a diagram for explaining the operation of the medical device and the applicator.

FIG. 45B is a front view of the hook viewed from direction B in FIG. 45A.

FIG. 45C is a cross-sectional view of the hook taken along line C-C in FIG. 45A.

FIG. 45D is a cross-sectional view of the hook taken along line D-D in FIG. 45A.

FIG. 46 is a diagram for explaining the operation of the medical instrument and the applicator.

### DETAILED DESCRIPTION

### First Embodiment

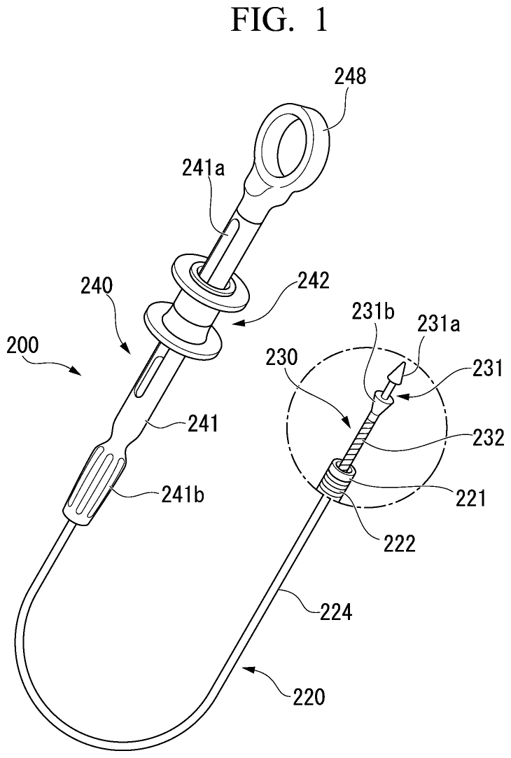

A first embodiment of the present disclosure will be described with reference to FIGS. 1 to 17.

A medical instrument 100 according to this embodiment is loaded into an applicator 200 that can be inserted through a channel of an endoscope 300 (see FIG. 9) and introduced to a treatment position.

[Applicator 200]

FIG. 1 is a perspective view of the applicator 200.

The applicator (medical instrument introduction device) 200 includes a sheath 220, an operation wire 230 and an operation portion 240. The applicator 200 is inserted through, for example, a treatment instrument insertion channel of the endoscope 300 and used in combination with the endoscope 300. Therefore, the sheath 220 is formed sufficiently longer than the treatment instrument insertion channel of the endoscope 300. The sheath 220 is flexible and curves according to the curvature of the insertion portion of the endoscope 300.

The sheath 220 includes a distal tip 221, a distal coil 222, and a proximal coil 224, and is formed in an elongated tubular shape as a whole. The distal coil 222 is arranged on the distal side of the sheath 220. The distal tip 221 is arranged at the distal end of the distal coil 222.

As shown in FIG. 1, the operation wire (power transmission member) 230 includes an arrowhead hook portion (connecting portion) 231 connected to the medical instrument 100 and a wire 232 for operating the arrowhead hook portion 231.

The arrowhead hook portion 231 includes a substantially conical engaging portion 231a that engages with the medical instrument 100, and a wire connecting portion 231b provided at the proximal end of the engaging portion 231a. The arrowhead hook portion 231 is made of, for example, a metal material such as stainless steel.

The wire 232 is inserted through the sheath 220 so as to be capable of being moved back and forth. The distal end of the wire 232 is fixed to the proximal end of the wire connecting portion 231b by welding, for example.

The operation portion 240 includes an operation portion main body 241, a slider 242, and a thumb ring 248, as shown in FIG. 1. The operation portion main body 241 is injection-molded, for example, from a resin material. The operation portion main body 241 includes a slit portion 241a and a rotary grip 241b on the distal end side. The slit portion 241a supports the slider 242 so as to be capable of being advanced and retracted.

The slider 242 is attached so as to be capable of being moved back and forth in the longitudinal axis direction of the operation portion main body 241, and the proximal end of the wire 232 is attached. As the slider 242 is advanced and retracted along the operation portion main body 241, the wire 232 is advanced and retracted with respect to the sheath 220, and the arrowhead hook portion 231 is advanced and retracted.

The thumb ring 248 is attached to the proximal end of the operation portion main body 241 so as to be capable of being rotated around the longitudinal axis of the operation portion main body 241.

### [Medical Instrument 100]

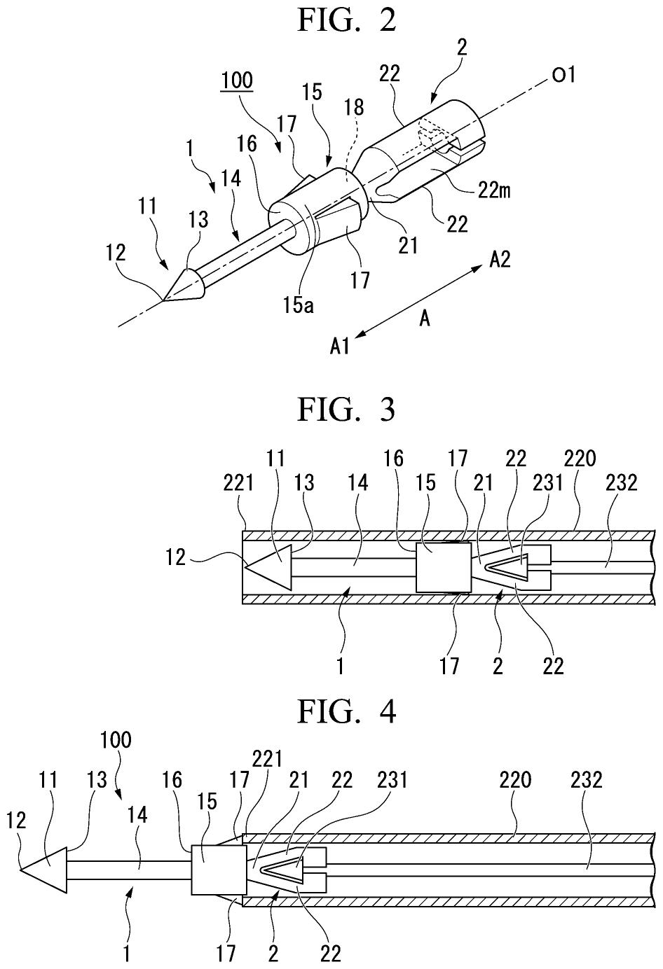

FIG. 2 is a perspective view of the medical instrument 100 according to this embodiment.

The medical instrument 100 includes an indwelling member 1 and a connecting member 2. The medical instrument 100 has a symmetrical shape with respect to the central axis O1 in the longitudinal direction A of the medical instrument 100.

In the following description, the side of the indwelling member 1 in the longitudinal direction A of the medical instrument 100 is referred to as the distal end side (distal side) A1 of the medical instrument 100, and the side of the connecting member 2 is referred to as the proximal end side (proximal side) A2 of the medical instrument 100.

The indwelling member 1 is formed in an arrowhead shape on the distal end side A1, and is a member that is punctured into living tissue by suturing. The indwelling member 1 includes a puncture portion 11, a rod-shaped portion 14, and a main body portion 15. The indwelling member 1 is made of a biocompatible material, such as stainless steel. The indwelling member 1 may be made of a bioabsorbable material.

The puncture portion 11 is provided on the distal end side A1 of the indwelling member 1 and is formed in a substantially conical shape. The puncture portion 11 has a sharp end portion 12 and a return portion 13. The distal end portion 12 is provided on the distal end side A1 of the puncture portion 11 and has a sharp distal end. The return portion 13 is provided on the proximal end side A2 of the puncture portion 11 and has the largest outer diameter in the puncture portion 11.

The rod-shaped portion 14 is formed in a cylindrical shape extending in the longitudinal direction A. The rod-shaped portion 14 is connected to the puncture portion 11 on the distal end side A1, and is connected to the main body portion 15 on the proximal end side A2. The outer diameter of the return portion 13 is larger than the outer diameter of the rod-shaped portion 14. That is, the outer diameter of the puncture portion 11 is larger than the outer diameter of rod-shaped portion 14 at the connecting portion between the puncture portion 11 and the rod-shaped portion 14.

The main body portion 15 is formed in a cylindrical shape extending in the longitudinal direction A. The main body portion 15 has an enlarged diameter portion 16, an engaging portion 17, and a connecting portion 18 connected to the connecting member 2.

The enlarged diameter portion 16 is provided on the distal end side A1 of the main body portion 15 and connected to the rod-shaped portion 14. The enlarged diameter portion 16 is formed in a planar shape in which the distal end side A1 is substantially perpendicular to the longitudinal direction A. The outer diameter of the enlarged diameter portion 16 is larger than the outer diameter of the rod-shaped portion 14. That is, the outer diameter of the main body portion 15 is larger than the outer diameter of the rod-shaped portion 14 at the connecting portion between the main body portion 15 and the rod-shaped portion 14.

For example, the length of the puncture portion 11 in the longitudinal direction A is approximately 5 mm. In addition, the length of the rod-shaped portion 14 in the longitudinal direction A (the length between the return portion 13 of the puncture portion 11 and the enlarged diameter portion 16 of the main body portion 15) is smaller than the spread width of a general clip, for example, about 10 mm.

The engaging portion 17 is a convex portion that protrudes from the outer peripheral surface 15a of the main body portion 15. The engaging portion 17 may be a pair of protrusions provided on both sides of the main body portion 15 with the central axis O1 interposed therebetween. The engaging portion 17 has a basic posture in which it protrudes radially outward with respect to the outer peripheral surface 15a. The engaging portion 17 receives a radially inward force from the outer side to enter a recessed state in which the engaging portion 17 is retracted with respect to the outer peripheral surface 15a. By releasing the above force, the engaging portion 17 returns from the recessed state to the projected state.

The connecting member 2 is separably connected to the connecting portion 18 of the indwelling member 1. In addition, the connecting member 2 is detachably connected to an arrowhead hook portion 231 inserted through the sheath 220. That is, the connecting member 2 connects the indwelling member 1 and the arrowhead hook portion 231. The connecting member 2 has a distal connecting portion 21 and a proximal connecting portion 22.

The distal connecting portion 21 is provided on the distal end side A1 of the connecting member 2, and the distal end portion is inserted into the internal space of the indwelling member 1 and connected to the connecting portion 18 in a separable manner. The distal connecting portion 21 is connected to the proximal connecting portion 22 on the proximal side A2.

The proximal end connecting portion 22 is detachably connected to the arrowhead hook portion 231 of the applicator 200. The proximal connecting portion 22 is bifurcated from the distal connecting portion 21 on the proximal side A2. The proximal connecting portion 22 is elastically deformable with respect to the distal connecting portion 21 and is capable of being opened and closed with respect to the distal connecting portion 21. A notch portion 22m is formed between the proximal end connecting portions 22 to hold and store the engaging portion 231a of the arrowhead hook portion 231. The cutout portion 22m is formed in a shape that closely contacts the outer peripheral surface of the engaging portion 231a of the arrowhead hook portion 231.

### [Operation and Action of Medical Instrument 100]

Next, the operation of the medical instrument 100 will be described. FIGS. 3 to 7 are diagrams for explaining the operation of the medical instrument 100.

The connecting member 2 of the medical instrument 100 loaded in the applicator 200 is connected to the arrowhead hook portion 231 that passes through the sheath 220, as shown in FIG. 3. The engaging portion 17 is pressed by the inner peripheral surface of the sheath 220 and is in a retracted state.

The operator introduces the medical instrument 100 loaded into the applicator 200 into the body through the channel of the endoscope 300. Next, the operator advances the arrowhead hook portion 231 by advancing the slider 242 along the operation portion main body 241. The operator advances the medical instrument 100 until the engaging portion 17 comes out of the sheath 220. As shown in FIG. 4, the engaging portion 17 transitions from the retracted state to the protruding state, which is the basic posture, by coming out of the sheath 220.

The engaging portion 17 in the projecting state engages with the distal tip 221 of the sheath 220 and is not pulled into the sheath 220. Therefore, the indwelling member 1 is not pulled into the sheath 220 after coming out of the sheath 220.

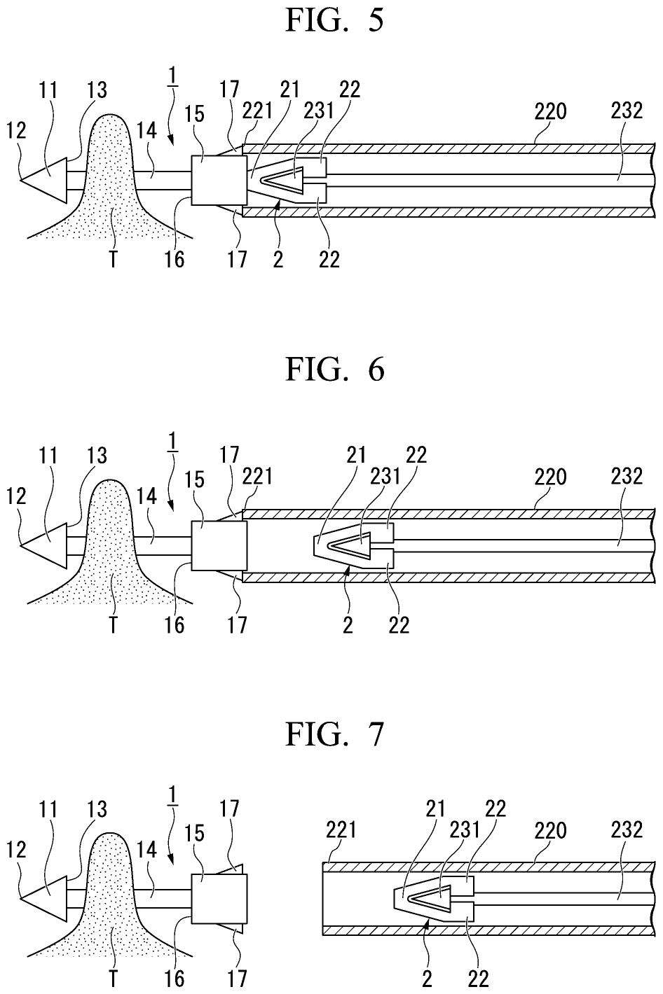

Next, the operator operates the endoscope 300 and the operation portion 240 to move the indwelling member 1 and puncture the living tissue T with the puncture portion 11, as shown in FIG. 5. The operator advances the indwelling member 1 until the return portion 13 of the puncture portion 11 penetrates the living tissue T. Since the engaging portion 17 engages with the distal tip 221 of the sheath 220, the operator can easily puncture the living tissue T with the puncture portion 11.

Next, the operator retracts the arrowhead hook portion 231 by retracting the slider 242 along the operation portion main body 241. The connecting member 2 connected to the arrowhead hook portion 231 pulls the indwelling member 1. Since the engaging portion 17 in the projecting state engages with the distal tip 221 of the sheath 220, it is not pulled into the sheath 220. As shown in FIG. 6, the distal end connecting portion 21 of the connecting member 2 is separated from the connecting portion 18 of the indwelling member 1 by, for example, partly breaking the distal end connecting portion 21. The connecting member 2 may be separated from the connecting portion 18 of the indwelling member 1 by elastic deformation or plastic deformation of a part of the distal end connecting portion 21, for example.

Next, as shown in FIG. 7, the operator retracts the endoscope 300 and the sheath 220, and indwells the indwelling member 1 that has punctured the living tissue T inside the body. In the indwelled indwelling member 1, the rod-shaped portion 14 sandwiched between the return portion 13 and the enlarged diameter portion 16 penetrates the living tissue T. The living tissue T is sandwiched between the return portion 13 and the enlarged diameter portion 16 that function to prevent coming off, and the indwelling member 1 does not come off from the living tissue T.

[Suture treatment using medical instrument 100]

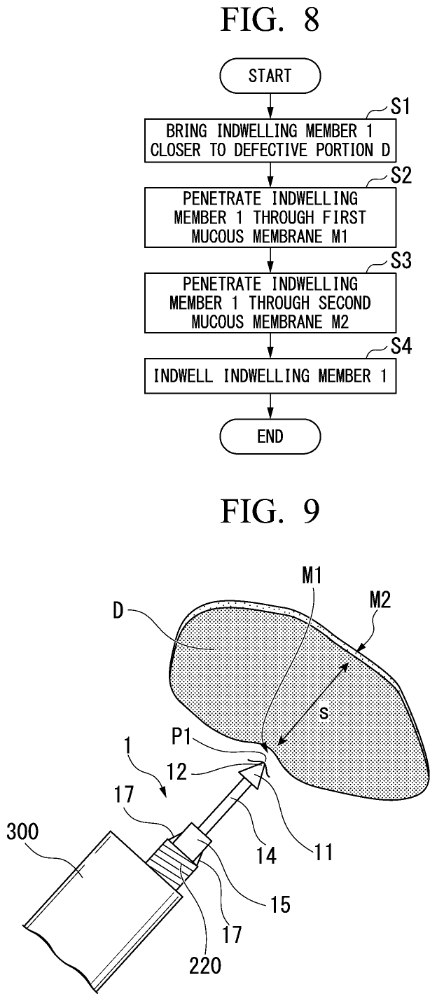

Next, suturing treatment using the medical instrument 100 will be described. Specifically, a treatment for suturing a defective portion D (diseased portion) formed in the body by endoscopic treatment will be described. FIG. 8 is a flow chart showing the suturing procedure.

### <First Step S1>

FIG. 9 is a diagram for explaining the first step S1 of suturing treatment.

First, the operator brings the indwelling member 1 closer to the defective portion D (first step S1). Henceforth, the content of the first step S1 is demonstrated concretely.

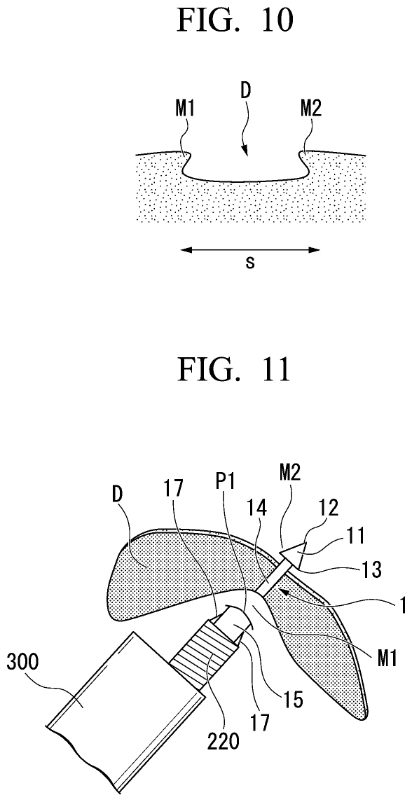

FIG. 10 is a cross-sectional view of the defective portion D in the suturing direction S.

The operator brings the endoscope 300 closer to the defective portion D. The operator observes the defective portion D and determines the suturing direction S in which the defective portion D is sutured based on the size and shape of the defective portion D. In the following description, the mucous membranes surrounding the defective portion D and arranged facing each other in the suturing direction S across the defective portion D are referred to as “first mucosa M1” and “second mucosa M2”.

The operator introduces the medical instrument 100 loaded into the applicator 200 into the body through the channel of the endoscope 300. As shown in FIG. 9, the operator moves the endoscope 300 and the sheath 220 to bring the indwelling member 1 of the medical instrument 100 closer to the first mucous membrane M1.

### <Second Step S2>

FIG. 11 is a diagram for explaining the second step S2 of the suture treatment.

The operator penetrates the indwelling member 1 through the first mucous membrane M1 (second step S2). Henceforth, the content of the second step S2 is demonstrated concretely.

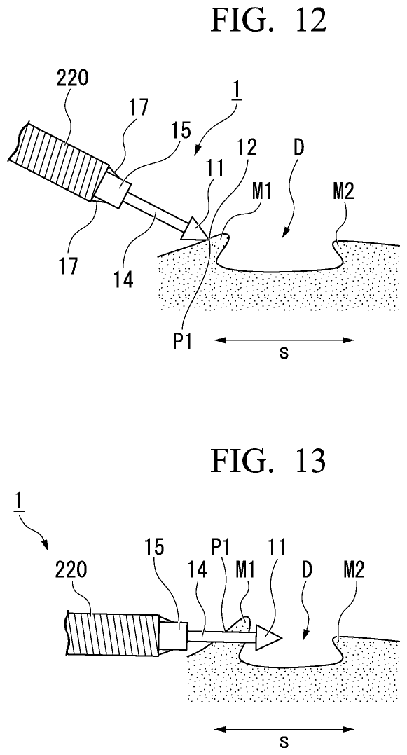

FIG. 12 is a cross-sectional view of the defective portion D in the second step S2, showing a state before the indwelling member 1 penetrates the first mucous membrane M1.

The operator presses the distal end portion 12 of the puncture portion 11 of the indwelling member 1 against the first portion P1 of the first mucous membrane M1 where the defective portion D has a large width in the suturing direction S. The operator creates a region where the puncture portion 11 of the indwelling member 1 can be easily punctured by pressing the distal end portion 12 against the first portion P1. At this time, the operator presses the indwelling member 1 against the mucous membrane M with the first strength. For example, the operator raises the first portion P1 as shown in FIG. 12. The operator may perform other treatments in order to create a region where the puncture portion 11 can be easily punctured. It should be noted that the operator may omit the treatment for creating the easy-puncture region if unnecessary.

FIG. 13 is a cross-sectional view of the missing portion D in FIG. 11.

The operator causes the indwelling member 1 to penetrate through the first portion P1 of the first mucous membrane M1 where the width of the defective portion D in the suturing direction S is large. Specifically, as shown in FIG. 11, the operator pierces the first portion P1 with the puncture portion 11 of the indwelling member 1 from the outside to the inside of the defective portion D, and pushes the indwelling member 1 until the puncture portion 11 of the indwelling member 1 penetrates the first portion P1. At this time, the operator pushes the indwelling member 1 with a second strength that is greater than the first strength.

### <Third Step S3>

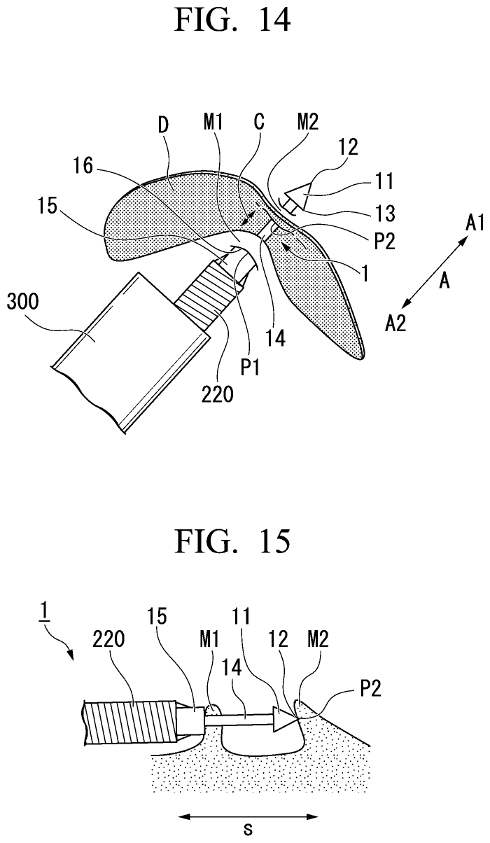

FIG. 14 is a diagram for explaining the third step S3 of suturing treatment.

The operator penetrates the indwelling member 1 through the second mucous membrane M2 (third step S3). Henceforth, the content of the third step S3 is demonstrated concretely.

FIG. 15 is a cross-sectional view of the defective portion D in the third step S3, showing the state before the indwelling member 1 penetrates the second mucous membrane M2.

The operator presses the distal end portion 12 of the puncture portion 11 of the indwelling member 1 against the second portion P2 of the second mucous membrane M2 where the defective portion D has a large width in the suturing direction S. The operator creates a region where the puncture portion 11 of the indwelling member 1 can be easily punctured by pressing the distal end portion 12 against the second portion P2. For example, the operator raises the second portion P2 as shown in FIG. 15. The operator may perform other treatments in order to create a region where the puncture portion 11 can be easily punctured. It should be noted that the operator may omit the treatment for creating the easy-puncture region if unnecessary.

The operator causes the indwelling member 1 to penetrate through the second portion P2 of the second mucous membrane M2 where the width of the defective portion D in the suturing direction S is large. Specifically, as shown in FIG. 14, the operator pierces the puncture portion 11 of the indwelling member 1 from the inside to the outside of the defective portion D to the second portion P2, and pushes the indwelling member 1 until the puncture portion 11 of the indwelling member 1 penetrates the second portion P2.

As shown in FIG. 14, in the indwelling member 1, the rod-shaped portion 14 sandwiched between the return portion 13 and the enlarged diameter portion 16 passes through the first portion P1 and the second portion P2. Therefore, the first portion P1 through which the indwelling member 1 penetrates cannot move toward the proximal side A2 from the enlarged diameter portion 16, and the second portion P2 through which the indwelling member 1 penetrates cannot move toward the distal side A1 from the return portion 13. Therefore, the first portion P1 and the second portion P2 through which the indwelling member 1 penetrates are arranged in the range between the return portion 13 and the enlarged diameter portion 16, and are arranged at positions closer to each other than before the treatment. The distance between the first portion P1 and the second portion P2 in the direction in which the indwelling member 1 extends (longitudinal direction A) becomes smaller than before the treatment.

From the viewpoint of suture treatment of a large defective portion D in a small number of treatment steps, it is desirable that the first portion P1 and the second portion P2 be portions where the width of the defective portion D in the suturing direction S is the largest.

The first portion P1 engages with the expanded diameter portion 16 to return to the proximal end side A2. The second portion P2 engages with the return portion 13 to return to the distal end side A1. Therefore, a gap C is formed between the first portion P1 and the second portion P2. However, if the length of the portion through which the indwelling member 1 penetrates at the first portion P1 and the second portion P2 is long, the gap C may not be formed.

### <Fourth Step S4>

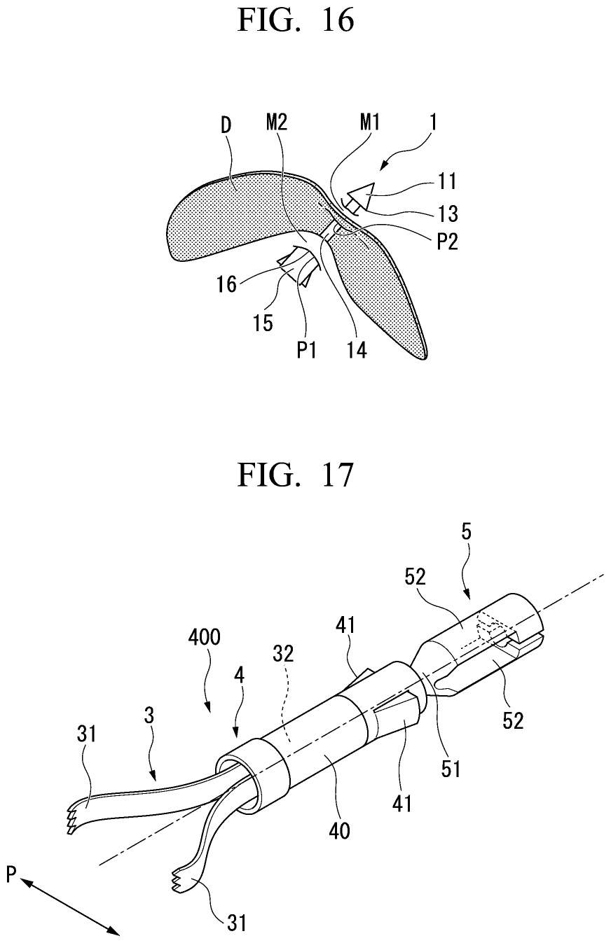

FIG. 16 is a diagram for explaining the fourth step S4 of the suture treatment.

The operator indwells the indwelling member 1 (fourth step S4). Henceforth, specific contents of the fourth step S4 will be described.

The operator pulls the connecting member 2 to the proximal side A2. The distal end connecting portion 21 of the connecting member 2 is separated from the connecting portion 18 of the indwelling member 1 by, for example, part of the distal end connecting portion 21 being broken or deformed. The operator withdraws the endoscope 300 and the sheath 220 to leave the indwelling member 1 in the body, which has passed through the first portion P1 and the second portion P2.

The operator pulls out the applicator 200 from the channel of the endoscope 300 to finish the suture treatment. The return portion 13 and the diameter-enlarged portion 16 functioning as slip-off prevention sandwich the first portion P1 and the second portion P2, and the indwelled indwelling member 1 does not slip out from the first portion P1 and the second portion P2.

According to the suturing treatment using the medical instrument 100 according to this embodiment, the treatment of suturing the defective portion D can be performed only by puncturing the indwelling member 1 into the first mucous membrane M1 and the second mucous membrane M2. Even if the defective portion D is large, which is difficult to suture with a clip, the defective portion D can be sutured in approximately one treatment. Therefore, suture treatment using the medical instrument 100 takes less time to suture a large defective portion D than suture treatment using a clip, stapler, or suture needle.

According to the suturing treatment using the medical instrument 100 according to this embodiment, it is sufficient to push the indwelling member 1 in the imaging direction, which is the longitudinal axis direction of the endoscope 300. Therefore, it is easy to adjust the position and attitude of the endoscope 300 (angle operation, etc.) so that the defective portion D, which is the treatment position, is within the field of view of the endoscope 300.

As described above, the first embodiment of the present disclosure has been described in detail with reference to the drawings, but the specific configuration is not limited to this embodiment, and design changes and the like are included within the scope of the present disclosure. Also, the constituent elements shown in the above-described embodiment and modifications shown below can be combined as appropriate.

### Second Embodiment

A second embodiment of the present disclosure will be described with reference to FIGS. 17 to 20. In the following description, the same reference numerals are given to the same configurations as those already described, and redundant descriptions will be omitted. The suturing treatment using the medical instrument 100 according to the second embodiment uses the clip unit 400 in addition to the medical instrument 100.

### [Clip Unit 400]

FIG. 17 is a perspective view of the clip unit 400.

The clip unit 400 is a known clip unit. Such a clip unit is disclosed in PCT International Publication No. WO 2021/172544, the entire text of which is incorporated herein by reference. The clip unit 400 includes, for example, a clip 3, a holding tube 4 as a tightening member, and a connecting member 5.

The clip (second indwelling member) 3 is formed by bending a metal plate material such as a leaf spring material made of stainless steel or the like at the center. The clip 3 has a pair of arms 31 that can be opened and closed in the opening and closing direction P, and a connecting portion 32 that connects the pair of arms 31.

The hold-down tube 4 has a hold-down tube main body 40 formed in a cylindrical shape and an engaging portion 41. The engaging portion 41 has the same function as the engaging portion 17 of the medical instrument 100.

The connecting member 5 is connected to the connecting portion 32 of the clip 3. In addition, the connecting member 5 is connected to the arrowhead hook portion 231 passing through the sheath 220 in the same manner as the connecting member 2 of the medical instrument 100. That is, the connecting member 5 connects the clip 3 and the arrowhead hook portion 231. The connecting member 5 has a distal connecting portion 51 inserted into the inner space of the pressing tube 4 and a proximal connecting portion 52 provided at the proximal end of the distal connecting portion 51.

The proximal connecting portion 52 is detachably connected to the arrowhead hook portion 231 of the applicator 200 in the same manner as the proximal connecting portion 22 of the medical instrument 100.

### [Suture Treatment Using Medical Instrument 100]

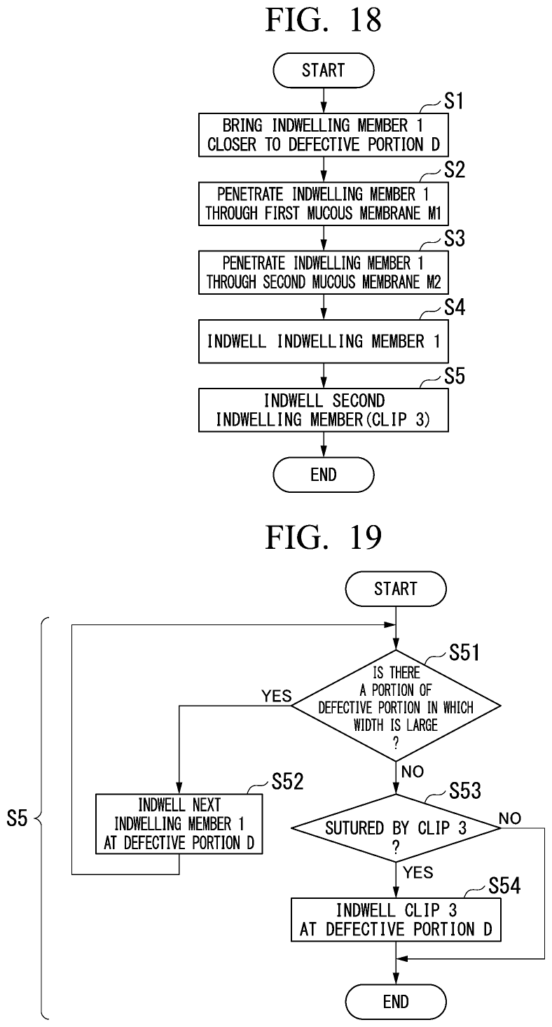

Next, suture treatment using the medical instrument 100 and the clip unit 400 will be described. FIG. 18 is a flow chart showing the suturing procedure. The first step S1 to the fourth step S4 in the suture treatment according to this embodiment are the same as in the first embodiment. The suturing treatment according to this embodiment further includes a fifth step S5.

### <Fifth Step S5>

FIG. 19 is a flow chart showing the procedure of the fifth step S5.

After the fourth step S4, the operator determines whether or not there is a portion of the defective portion D in which the width in the suturing direction S is so large that it cannot be sutured with the medical clip 3, except for the sites (the first portion P1 and the second portion P2) where the suturing treatment is performed using the indwelling member 1 of the medical instrument 100 (step S51).

If there is a portion that cannot be sutured with the clip 3, the operator sutures the portion using the medical instrument 100 in the same manner as in the first embodiment (step S52). The length of the rod-shaped portion 14 of the medical instrument 100 in the longitudinal direction A (the length between the return portion 13 of the puncture portion 11 and the enlarged diameter portion 16 of the main body portion 15) is shorter than the maximum opening width of the pair of arms 31 of the clip 3. Therefore, by using the indwelling member 1 of the medical instrument 100 to reduce the width of the defective portion D in the suturing direction S, the operator can easily perform suturing treatment by using the pair of arms 31 of the clip 3 at portions other than the sutured portion using the indwelling member 1 of the medical instrument 100. The operator punctures the mucous membrane surrounding the defective portion D with the second medical instrument 100 (indwelling member 1) in the same direction as the first medical instrument 100 (indwelling member 1) has been punctured. As a result, the operator can puncture the mucous membrane surrounding the defective portion D with the second medical instrument 100 (indwelling member 1) without significantly changing the position and bending angle of the distal end of the endoscope 300.

A portion of the first mucous membrane M1 other than the first portion P1 is referred to as a “third portion P3”. Also, a portion of the second mucous membrane M2 other than the second portion P2 and facing the third portion P3 in the suturing direction S is referred to as a “fourth portion P4”. When the length between the third portion P3 and the fourth portion P4 is longer than the length that can be sutured by the clip 3, the operator penetrates the second indwelling member 1 through the third portion P3 and the fourth portion P4 to be indwelled.

If there is no third portion P3 or fourth portion P4 such that the length between the third portion P3 and the fourth portion P4 is longer than the length that can be sutured by the clip 3, the operator 3 determines whether there is a portion to be sutured (step S53).

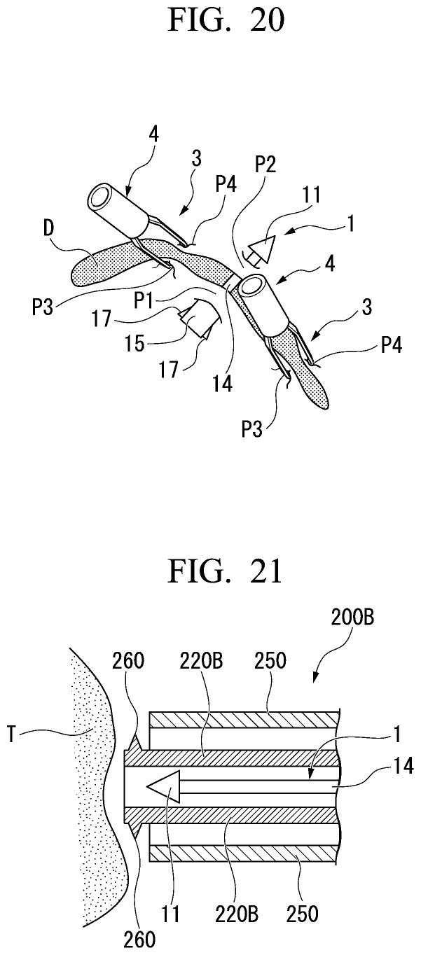

FIG. 20 is a diagram for explaining the fifth step S5 of the suturing treatment.

When there is a portion to be sutured using the clip 3, the operator sutures the portion using the clip unit 400 by a known method, as shown in FIG. 20 (step S54). When the length between the third portion P3 and the fourth portion P4 is a length that can be sutured with the clip 3, the operator grasps the third portion P3 and the fourth portion P4 with the clip 3 and pulls them together, and indwells the clip 3 there.

According to the suturing treatment using the medical instrument 100 and the clip unit 400 according to this embodiment, by using different treatment tools (the medical instrument 100 and the clip unit 400) based on the width of the defective portion D in the suturing direction S, the suturing treatment of suturing a plurality of sites can be performed more appropriately.

As described above, the second embodiment of the present disclosure has been described in detail with reference to the drawings, but the specific configuration is not limited to this embodiment, and design changes and the like are also included within the scope of the present disclosure. Also, the constituent elements shown in the above-described embodiment and modifications shown below can be combined as appropriate.

### (Modification)

In the above embodiment, the clip 3 is used as the second indwelling member in the fifth step S5, but the fifth step S5 is not limited to suture treatment using the clip 3. The fifth step S5 may be suturing using a thread as the second indwelling member and suturing with a thread and a needle.

### Third Embodiment

A third embodiment of the present disclosure will be described with reference to FIGS. 21 to 25. In the following description, the same reference numerals are given to the same configurations as those already described, and redundant descriptions will be omitted. The suturing treatment using the medical instrument 100 according to the third embodiment uses an applicator 200B that is a modification of the applicator 200.

### [Applicator 200B]

The applicator 200B differs from the applicator 200 of the first embodiment in that the sheath has a double structure. The applicator 200B includes a sheath 220B, an outer sheath 250, an operation wire 230, and an operation portion 240.

FIG. 21 is a cross-sectional view of the distal end of the sheath 220B.

The sheath (inner sheath) 220B is a coil sheath similar to the sheath 220 of the first embodiment. A distal tip 221 at the distal end of the sheath 220B is provided with a projection 260 facing radially outward. The protrusions 260 may be protrusions provided at two portions facing each other in the radial direction, or may be protrusions provided along the entire circumference in the circumferential direction.

The outer sheath 250 is a sheath provided on the outer peripheral side of the sheath 220B. The sheath 220B and the outer sheath 250 can be advanced and retracted independently by the operation portion 240.

### [Suture Treatment Using Medical Instrument 100 and Applicator 200B]

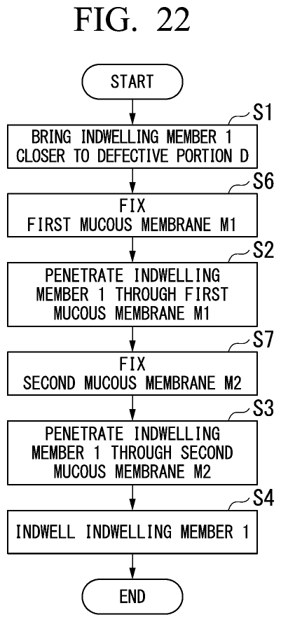

Next, suture treatment using the medical instrument 100 and the applicator 200B will be described. FIG. 22 is a flow chart showing the suturing procedure. The first step S1 to the fourth step S4 in the suture treatment according to this embodiment are the same as in the first embodiment. The suturing treatment according to this embodiment further includes a first auxiliary step of fixing the periphery of the first portion P1 before the second step S2, and a second auxiliary step of fixing the periphery of the second portion P2 before the third step S3.

### <First Auxiliary Step S6>

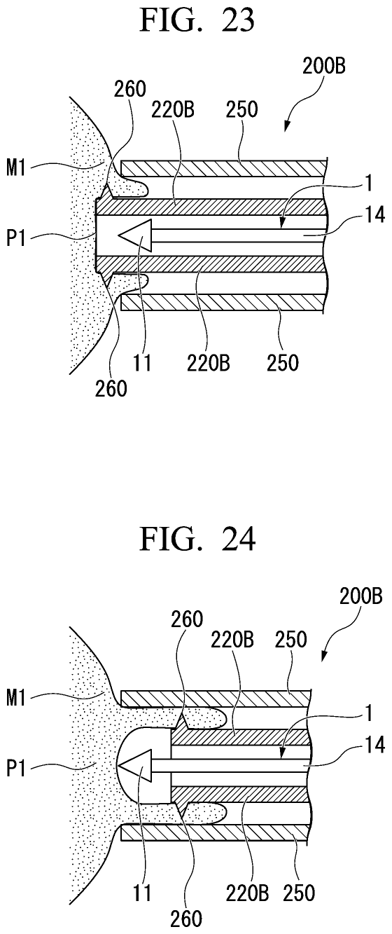

FIGS. 23 and 24 are diagrams for explaining the first auxiliary step S6.

Before the second step S2, the operator fixes the first mucous membrane M1 around the first portion P1 (first auxiliary step S6). Specifically, as shown in FIG. 23, the operator advances the sheath 220B and presses it against the first mucous membrane M1 around the first portion P1. Next, the operator advances the outer sheath 250 to sandwich the first mucous membrane M1 between the sheath 220B and the outer sheath 250. Since the projection 260 is hooked on the first mucous membrane M1, the operator can easily sandwich the first mucous membrane M1 between the sheath 220B and the outer sheath 250.

Next, as shown in FIG. 24, the operator causes the indwelling member 1 to penetrate through the first portion P1 (second step S2). Since the first mucous membrane M1 is sandwiched and fixed between the sheath 220B and the outer sheath 250, the operator can easily puncture the first portion P1 with the puncture portion 11 of the indwelling member 1.

### <Second Auxiliary Step S7>

The second auxiliary step S7 is the same as the first auxiliary step S6 except that the treatment is performed not at the second portion P1 but at the second portion P2.

According to the suturing treatment using the medical instrument 100 and the applicator 200B according to this embodiment, the puncture portion 11 of the indwelling member 1 can easily puncture the first portion P1 and the second portion P2.

As described above, the third embodiment of the present disclosure has been described in detail with reference to the drawings, but the specific configuration is not limited to this embodiment, and design changes and the like are included within the scope of the present disclosure. Also, the constituent elements shown in the above-described embodiment and modifications shown below can be combined as appropriate.

### (Modification)

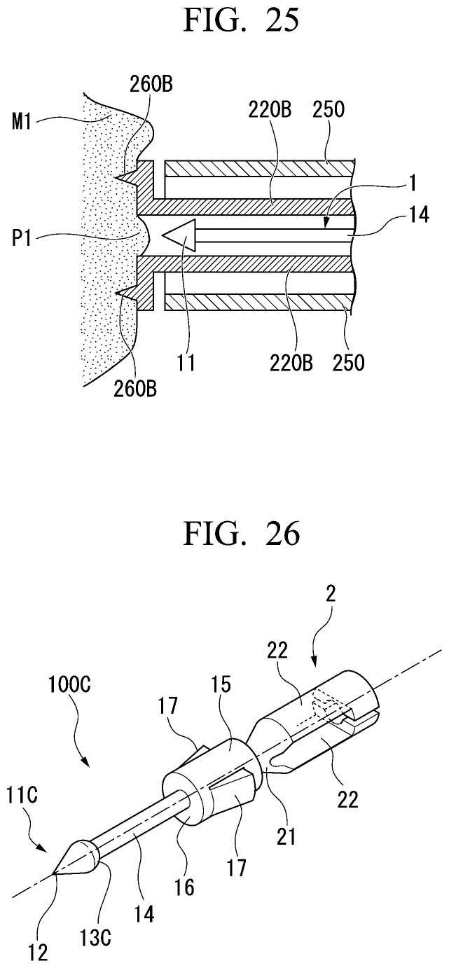

For example, in the above embodiment, the protrusion 260 faces radially outward. However, the aspect of the protrusions provided on the sheath 220B is not limited to this. FIG. 25 shows a projection 260B that is a modified example of projection 260. The protrusion 260B is provided so as to face the distal end side. The protrusions 260B may be protrusions provided at two portions facing each other in the radial direction, or may be protrusions provided along the entire circumference in the circumferential direction. In this case, the outer sheath 250 is not necessarily required.

### Fourth Embodiment

A fourth embodiment of the present disclosure will be described with reference to FIGS. 26 to 35. In the following description, the same reference numerals are given to the same configurations as those already described, and redundant descriptions will be omitted. Compared with the medical instrument 100 of the first embodiment, the medical instrument 100C and the like according to the fourth embodiment are easier to remove the indwelling member 1 that has punctured the mucous membrane and to re-insert the indwelling member 1.

FIG. 26 is a perspective view of a medical instrument 100C according to this embodiment.

The medical instrument 100C includes a puncture portion 11C that is a modification of the puncture portion 11. The puncture portion 11C has a sharp distal end portion 12 with a sharp distal end and a return portion 13C. The return portion 13C is formed in the shape roundish compared with the return portion 13 of the first embodiment. Therefore, when moving the medical instrument 100C that has punctured the mucous membrane to the proximal end side, the puncture portion 11C is more likely to come off the mucous membrane than the puncture portion 11 of the first embodiment.

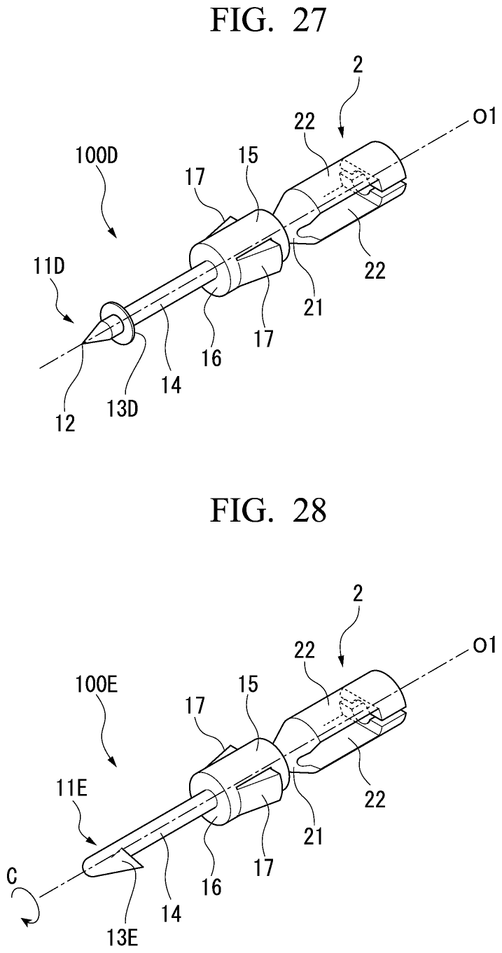

FIG. 27 is a perspective view of a medical instrument 100D according to another aspect of this embodiment.

The medical instrument 100D includes a puncture portion 11D that is a modification of the puncture portion 11. The puncture portion 11D has a sharp distal end portion 12 with a sharp distal end and a return portion 13D. The return portion 13D is formed in a disc shape by an elastic member. Since the return portion 13D is elastically deformable, when the medical instrument 100D that has punctured the mucous membrane is moved to the proximal end side, the puncture portion 11D is more easily removed from the mucous membrane than the puncture portion 11 of the first embodiment.

FIG. 28 is a perspective view of a medical instrument 100E according to another aspect of this embodiment.

The medical instrument 100E includes a puncture portion 11E that is a modification of the puncture portion 11. The puncture portion 11E has a sharp distal end portion 12 with a sharp distal end and a return portion 13E. The return portion 13E is not provided over the entire circumference in the circumferential direction C, unlike the return portion 13 of the first embodiment, but is provided only partially in the circumferential direction C.

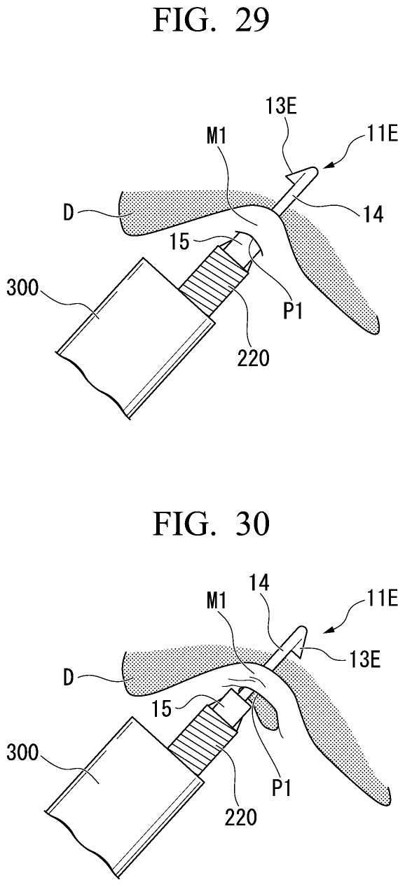

FIGS. 29 and 30 are diagrams for explaining treatment by using the medical instrument 100E.

As shown in FIG. 29, the operator can move the position of the return portion 13E by rotating the puncture portion 11E in the circumferential direction C even after the puncture portion 11E has penetrated the first mucous membrane M1. As shown in FIG. 30, the operator moves the return portion 13E to a position where it can easily pass through the hole formed in the first mucosa M1, thereby facilitating removal of the puncture portion 11E from the first mucosa M1.

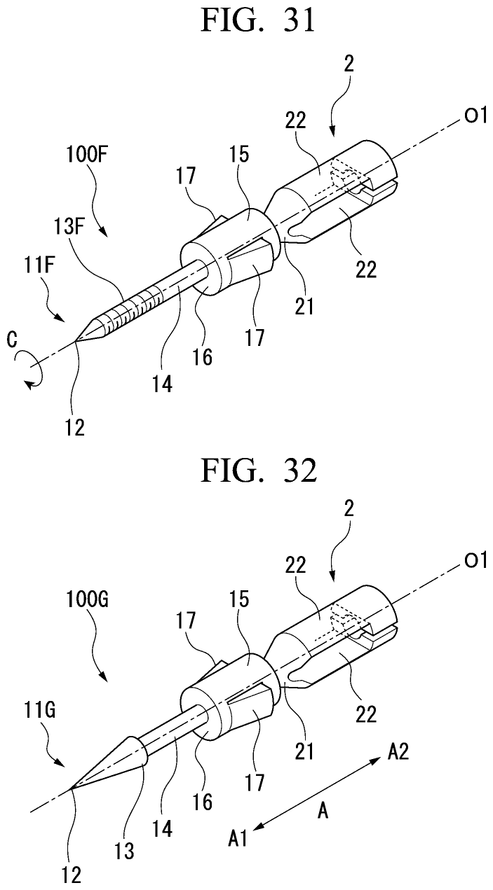

FIG. 31 is a perspective view of a medical instrument 100F according to another aspect of this embodiment.

The medical instrument 100F includes a puncture portion 11F that is a modification of the puncture portion 11. The puncture portion 11F has a sharp end portion 12 with a sharp distal end and a screw portion 13F. The screw portion 13F is a screw thread formed along the circumferential direction C in a screw shape. By rotating the screw portion 13F in the circumferential direction C, the operator can easily remove the puncture portion 11F from the mucous membrane.

FIG. 32 is a perspective view of a medical instrument 100G according to another aspect of this embodiment.

The medical instrument 100G includes a puncture portion 11G that is a modification of the puncture portion 11. The puncture portion 11G has the same shape as the puncture portion 11 of the first embodiment, but the dimension in the longitudinal direction A is different. The puncture portion 11G has a longer length in the longitudinal direction A than the puncture portion 11 of the first embodiment. The puncture portion 11G has a smaller inclination angle with respect to the central axis O1 than the puncture portion 11 of the first embodiment. Therefore, the puncture portion 11G is more likely to come out of the mucosa than the puncture portion 11 of the first embodiment. For example, the length in the longitudinal direction A of the puncture portion 11G is approximately 10 mm, and the length in the longitudinal direction A of the rod-shaped portion 14 is approximately 10 mm. Since the length of the puncturing portion 11G in the longitudinal direction A is long, when the puncturing portion 11G is punctured into the mucous membrane, it is likely that the distal end portion 12 penetrates the mucous membrane, but the return portion 13 does not penetrate the mucous membrane. The operator can easily confirm the puncture position by looking at the distal end portion 12 that has penetrated the mucous membrane. Furthermore, even when the operator changes the confirmed puncture position, the operator can easily pull out the puncture portion 11G from the mucous membrane because the return portion 13 does not penetrate the mucous membrane.

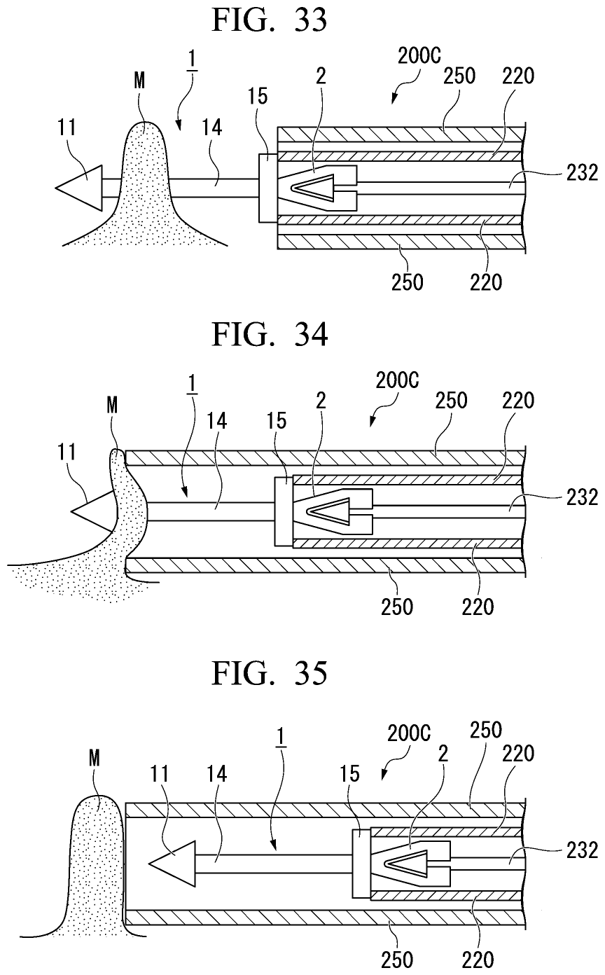

FIGS. 33 to 35 are diagrams for explaining a suturing treatment according to another aspect of this embodiment. A medical instrument 100 and an applicator 200C are used for suture treatment in this mode.

The applicator 200C differs from the applicator 200 of the first embodiment in that the sheath has a double structure. The applicator 200B includes a sheath 220, an outer sheath 250, an operation wire 230, and an operation portion 240.

The operator pierces the mucous membrane M with the puncture portion 11 of the indwelling member 1 as shown in FIG. 33. Next, the operator advances the outer sheath 250 with respect to the sheath 220 as shown in FIG. 34. Next, the operator retracts the indwelling member 1 as shown in FIG. 35. Since the mucous membrane M is engaged with the distal end of the outer sheath 250, the operator can easily remove the puncture portion 11 from the mucous membrane M. Even if the indwelling member 1 is retracted without advancing the outer sheath 250, since the mucous membrane M retracted together with the indwelling member 1 engages with the distal end of the outer sheath 250, the operator can easily remove the puncture portion 11 from the mucous membrane M.

According to the suturing treatment using the medical instrument 100C or the like and the applicator 200C according to this embodiment, it is easy to remove the indwelling member 1 that has punctured the mucous membrane. Therefore, the operator can easily perform the following treatment. After penetrating the indwelling member 1 through the first portion P1 of the first mucous membrane M1, the operator pulls out the indwelling member 1 from the first portion P1 and allows the indwelling member 1 to penetrate through another portion of the first mucous membrane M1. Further, after penetrating the indwelling member 1 through the second portion P2 of the second mucous membrane M2, the operator pulls out the indwelling member 1 from the second portion P2 and penetrates the indwelling member 1 through another portion of the second mucous membrane M2.

As described above, the fourth embodiment of the present disclosure has been described in detail with reference to the drawings, but the specific configuration is not limited to this embodiment, and design changes and the like are included within the scope of the present disclosure. Also, the constituent elements shown in the above-described embodiment and modifications shown below can be combined as appropriate.

### Fifth Embodiment

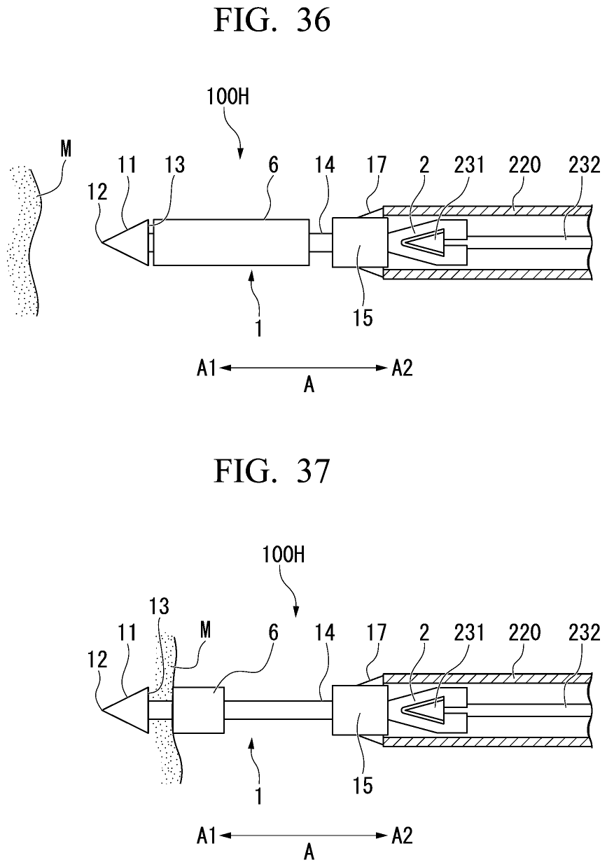

A fifth embodiment of the present disclosure will be described with reference to FIGS. 36 to 39. In the following description, the same reference numerals are given to the same configurations as those already described, and redundant descriptions will be omitted. The medical instrument 100H according to the fifth embodiment includes a regulating member that regulates the amount of puncture into the mucous membrane compared to the medical instrument 100 of the first embodiment.

FIGS. 36 and 37 are diagrams showing a medical instrument 100H according to this embodiment.

The medical instrument 100H further includes an elastic member 6 compared to the medical instrument 100 of the first embodiment.

The elastic member (regulating member) 6 is formed in a cylindrical shape from an elastic material such as rubber. The elastic member 6 surrounds the outer circumference of the rod-shaped portion 14. The distal end of the elastic member 6 engages with the return portion 13. When the operator pushes the indwelling member 1 against the mucous membrane M, a portion of the mucous membrane M enters between the return portion 13 and the elastic member 6 by restoring the mucous membrane M in the direction of closing the hole formed in the mucous membrane M as shown in FIG. 37. Therefore, even if the operator tries to further push the indwelling member 1 into the mucous membrane M, the indwelling member 1 cannot be pushed out. As a result, the amount of puncturing of the mucous membrane M by the indwelling member 1 is regulated.

According to the suturing treatment using the medical instrument 100H according to the present embodiment, the amount of puncturing of the mucous membrane by the indwelling member 1 can be regulated, and it is possible to prevent damage to the muscle layer or the like due to excessive puncturing of the indwelling member 1.

As described above, the fifth embodiment of the present disclosure has been described in detail with reference to the drawings, but the specific configuration is not limited to this embodiment, and design changes and the like are included within the scope of the present disclosure. Also, the constituent elements shown in the above-described embodiment and modifications shown below can be combined as appropriate.

### (Modification)

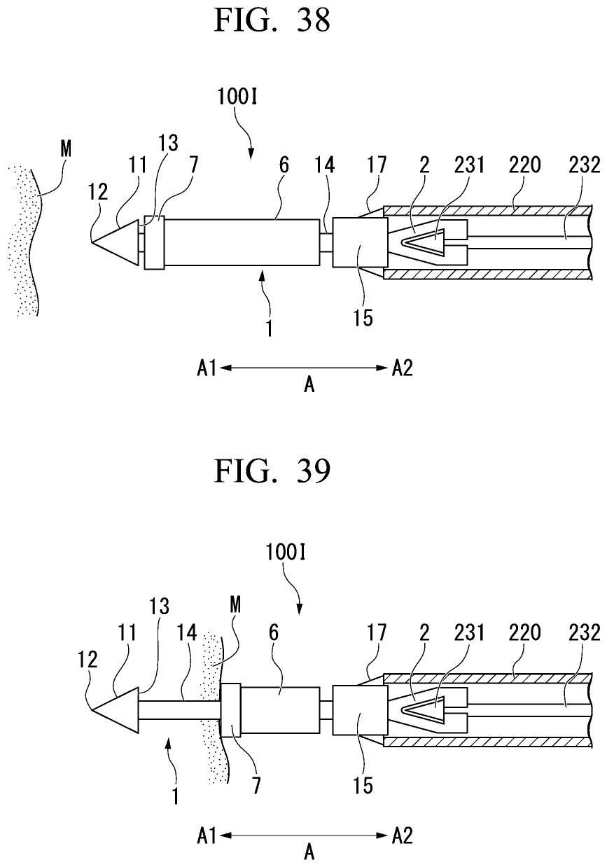

For example, the medical instrument 100H may have the stopper 7 in the above embodiment. FIGS. 38 and 39 show a medical instrument 1001 further including a stopper 7. A stopper 7 is provided at the distal end of the elastic member 6. When the operator pushes the indwelling member 1 of the medical instrument 1001 against the mucous membrane M, the stopper 7 is pushed by the mucous membrane M and moves to the proximal side A2. As shown in FIG. 39, the elastic member 6 is compressed by the mucous membrane pushing the stopper 7 toward the proximal end side A2. The elastic member 6 functions like a damper to prevent the indwelling member 1 from penetrating the mucous membrane too much.

### Sixth Embodiment

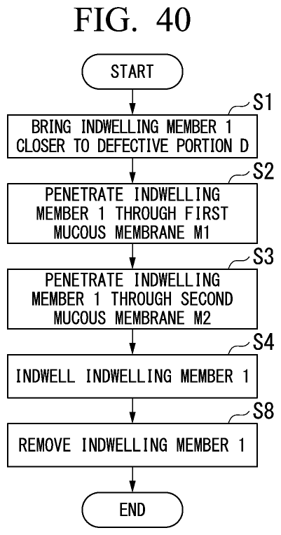

A sixth embodiment of the present disclosure will be described with reference to FIG. 40. In the following description, the same reference numerals are given to the same configurations as those already described, and redundant descriptions will be omitted.

A suturing treatment using the medical instrument 100J according to this embodiment will be described. FIG. 40 is a flow chart showing the suturing procedure. The first step S1 to the fourth step S4 in the suture treatment according to this embodiment are the same as in the first embodiment. The suturing treatment according to this embodiment further includes a removing step S8.

### <Removal Step S8>

The medical instrument 100J further includes a fragile portion 8 that is easily destroyed by an external force or the like, compared to the medical instrument 100 of the first embodiment. The operator destroys the fragile portion 8 of the indwelled medical instrument 100J and removes the medical instrument 100J from the defective portion D (removal step S8).

The fragile portion 8 has mechanical fragility, for example. The fragile portion 8 having mechanical fragility is, for example, a portion that is locally formed to be thinner than other portions, or a portion that is provided with a mechanically fragile portion by heat treatment or the like.

The fragile portion 8 has, for example, electrical fragility. The fragile portion 8 having electrical fragility is, for example, a portion that is locally formed to be thinner than other portions, and is a portion that is likely to break when an electric current flows from the outside.

The fragile portion 8 has, for example, chemical fragility. The fragile portion 8 having chemical fragility is, for example, a portion that is likely to break when exposed to body fluids. For example, when the indwelling member 1 is made of a magnesium alloy and is entirely covered with an oxide film, the fragile portion 8 having chemical fragility is a portion where the base material of magnesium is exposed by partially removing the surface of the oxide film. By increasing the exposed area of the magnesium matrix, the chemical vulnerability can be increased.

The fragile portion 8 is provided in the return portion 13, for example. The fragile portion 8 may be formed in the return portion 13 by removing the oxide film of the return portion 13. Further, the return portion 13 may be formed with a hole or a spiral groove to increase the exposed area of the magnesium base material, thereby increasing mechanical and chemical fragility.

When the fragile portion 8 formed in the return portion 13 is destroyed, the mucous membrane sandwiched between the return portion 13 and the enlarged diameter portion 16 can move to the distal end side A1 from the return portion 13. Therefore, the indwelling member 1 that has been indwelled can be easily removed from the mucous membrane.

The weakened portion 8 is provided, for example, on the rod-shaped portion 14. The weakened portion 8 may be formed in the rod-shaped portion 14 by removing the oxide film of the rod-shaped portion 14. Furthermore, by forming a hole or forming a spiral groove in the rod-shaped portion 14, the exposed area of the magnesium base material may be increased to increase mechanical and chemical fragility.

When the fragile portion 8 formed in the rod-shaped portion 14 is destroyed, the indwelling member 1 is separated into two parts, the distal side including the return portion 13 and the proximal side including the enlarged diameter portion 16. Therefore, the portion on the distal end side including the return portion 13 and the portion on the proximal end side including the enlarged diameter portion 16 are easily removed from the mucous membrane.

The fragile portion 8 is provided, for example, at the distal end portion 12. The weakened portion 8 may be formed in the distal end portion 12 by removing the oxide film of the distal end portion 12. Furthermore, a hole or a spiral groove may be formed in the sharp end 12 to increase the exposed area of the magnesium base material, thereby increasing mechanical and chemical fragility.

By destroying the fragile portion 8 formed in the distal end portion 12, it is possible to prevent tissue from being unintentionally damaged by the distal end portion 12 when the indwelling member 1 remains in the body for a long period of time.

According to the suturing treatment using the medical instrument 100J according to this embodiment, the indwelling member 1 that is no longer needed can be easily removed from the body. In addition, it is possible to prevent the tissue from being unintentionally damaged by the distal end portion 12.

As described above, the sixth embodiment of the present disclosure has been described in detail with reference to the drawings, but the specific configuration is not limited to this embodiment, and design changes and the like are included within the scope of the present disclosure. Also, the constituent elements shown in the above-described embodiment and modifications shown below can be combined as appropriate.

### Seventh Embodiment

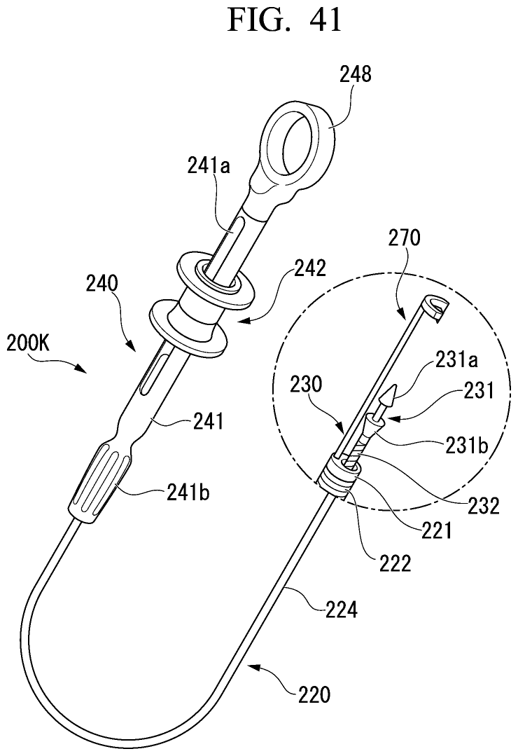

A seventh embodiment of the present disclosure will be described with reference to FIGS. 41 to 46. In the following description, the reference numerals are given to the configurations as those already described, and redundant descriptions will be omitted.

A medical instrument 100K according to this embodiment is loaded into an applicator 200K that can be inserted through a channel of an endoscope 300 (see FIG. 9) and introduced to a treatment position.

### [Applicator 200K]

FIG. 41 is a perspective view of the applicator 200K.

The applicator (medical instrument introduction device) 200K includes a sheath 220, an operation wire 230, an operation portion 240, and a hook 270. The applicator 200K is inserted through, for example, a treatment instrument insertion channel of the endoscope 300 and used in combination with the endoscope 300.

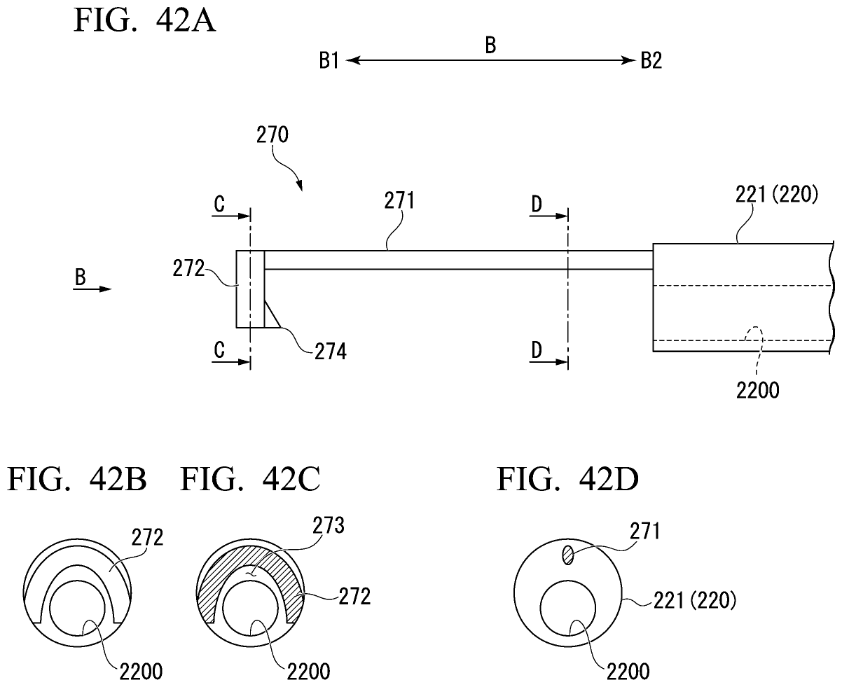

FIG. 42A is a side view of the hook 270. FIG. 42B is a front view of hook 270 viewed from direction B in FIG. 42A. FIG. 42C is a cross-sectional view of hook 270 along line C-C in FIG. 42A. FIG. 42D is a cross-sectional view of hook 270 along line D-D in FIG. 42A.

A hook (holding member) 270 is arranged on the distal end side of the distal tip 221. The hook 270 includes a hook main body 271 and a hook distal end portion (restriction portion) 272. In the following description, the side of the hook distal end portion 272 in the longitudinal direction B of the hook 270 is referred to as the distal end side (distal side) B1 of the hook 270, and the side of the sheath 220 is referred to as the proximal end side (proximal side) B2 of the hook 270.

The hook main body 271 is a member extending along the longitudinal direction B. The hook main body 271 may have a cross-sectional shape having a first axis (major axis) and a second axis (minor axis) shorter than the first axis in a cross section perpendicular to the longitudinal direction B. The cross-sectional shape of the hook main body 271 is, for example, elliptical, oval, or rectangular. In this case, the hook main body 271 is hard to bend in the first axial direction and easy to bend in the second axial direction.

The hook distal end portion 272 is provided on the distal end side B1 of the hook main body 271. The hook distal end portion 272 has a passageway 273 that, in cross-section perpendicular to longitudinal direction B, is larger than the outer diameter of channel 2200 of the sheath 220. For example, the hook distal end portion 272 is formed in a substantially U shape when viewed from the longitudinal direction B. The hook distal end portion 272 may have an anchor 274. For example, the anchor 274 is a protrusion facing the proximal end side B2 of the hook. A plurality of anchors 274 may be provided at the hook distal end portion 272. In addition, the anchor 274 may be a surface having a higher coefficient of friction than other portions provided on the proximal end side B2 of the hook distal end portion 272.

The hook main body 271 may be detachably connected to the sheath 220. Specifically, the hook main body 271 of the hook 251 may be detachably connected to the distal tip 221 of the sheath 220. When loading the medical instrument 100K and the clip unit 400 from the distal end side B1 of the applicator 200K, by removing the hook main body 271 from the sheath 220, the medical instrument 100K and the clip unit 400 can be smoothly loaded into the applicator 200K.

The slider 242 may be provided with a configuration for informing the user of rotation of the wire 232 and the medical device 100K connected to the wire 232 around the longitudinal direction A. Specifically, the slider 242 and the operation unit main body 241 are provided with unevenness and colored markers, so that the user can visually recognize the rotation of the medical device 100K around the longitudinal direction A and adjust the position of the medical device 100K.

### [Medical Instruments 100K]

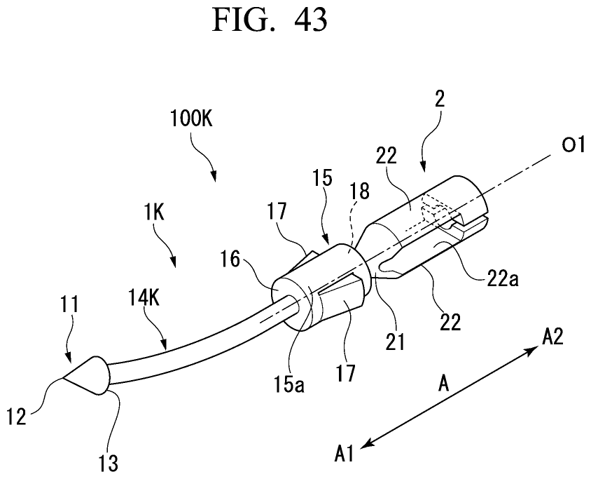

FIG. 43 is a perspective view of the medical device 100K.

The medical device 100K includes an indwelling member 1K and a connecting member 2.

The indwelling member 1K has an arrowhead-shaped distal end A1, and is a member that is punctured into living tissue by suturing. The indwelling member 1K includes a puncturing portion (distal portion) 11, a rod-shaped portion (intermediate portion) 14K, and a body portion (base end portion) 15. The indwelling member 1K is made of a biocompatible material, such as stainless steel. The indwelling member 1K may be made of a bioabsorbable material such as polylactic acid.

The puncturing portion 11 is provided on the distal end side A1 of the indwelling member 1K and is formed in a substantially conical shape. The puncturing portion 11 has a distal end portion (first portion) 12 and a return portion (second portion) 13. The distal end portion 12 is provided on the distal end side A1 of the puncturing portion 11 and has a sharp distal end. The return portion 13 is provided on the proximal end side A2 of the puncturing portion 11 and has the largest outer diameter in the puncturing portion 11.

The rod-shaped portion 14K is formed in a substantially cylindrical shape extending in the longitudinal direction A. The rod-shaped portion 14K is connected to the puncturing portion 11 on the distal end side A1, and is connected to the body portion 15 on the proximal end side A2. The outer diameter of the return portion 13 is larger than the outer diameter of the rod-shaped portion 14K. That is, at the connecting portion between puncturing portion 11 and rod-shaped portion 14K, the outer diameter of puncturing portion 11 is larger than the outer diameter of rod-shaped portion 14K.

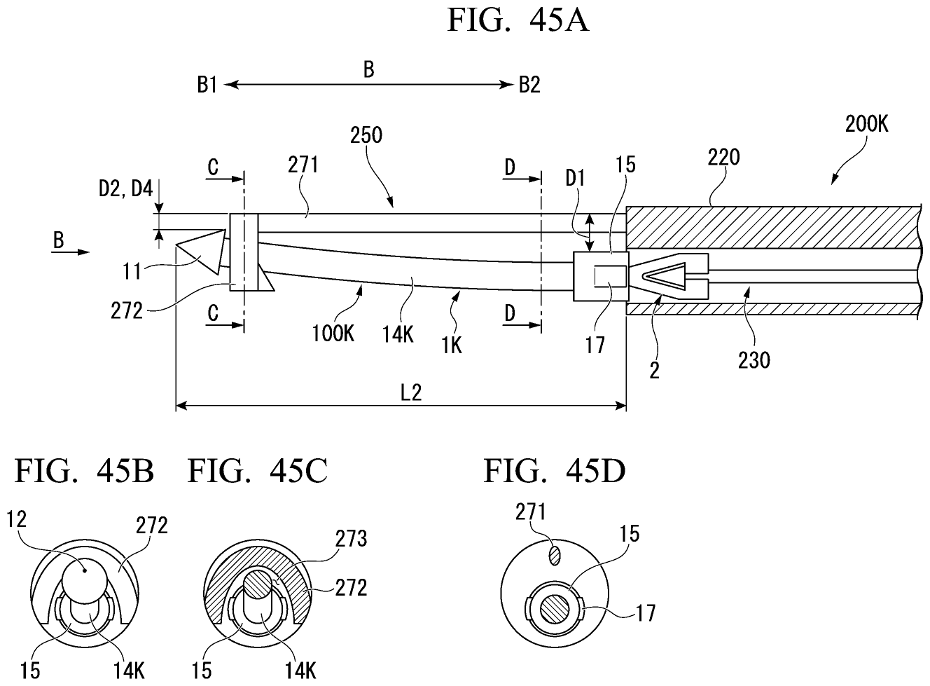

The rod-shaped portion 14K is curved in the longitudinal direction A. Specifically, the intermediate portion curves in the radial direction of the sheath 220 in the direction in which the puncturing portion 11 approaches the hook 270. That is, as shown in FIG. 45A, in a state in which the indwelling member 1K is arranged outside the sheath 220, the distance (second distance D2) between the puncturing portion 11 and the hook 270 in the radial direction of the sheath 220 is smaller than the distance (first distance D1) between the body portion 15 and the hook 270.

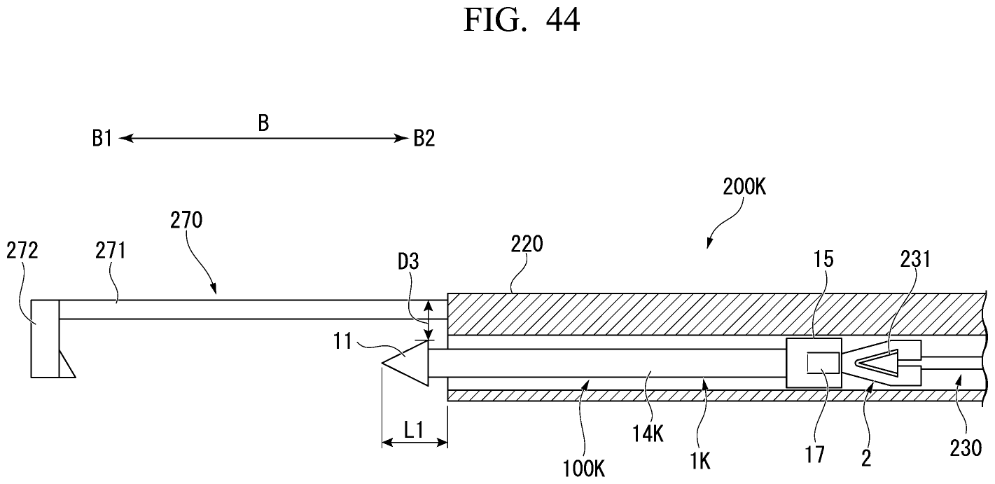

As shown in FIGS. 44 and 45A, the distance (third distance D3) between the puncturing portion 11 and the hook 270 in a state (first state) in which the indwelling member 1K protrudes from the sheath 220 by a first length L1 is greater than the distance (fourth distance D4) between puncturing portion 11 and hook 270 in a state (second state) in which the indwelling member 1K protrudes from sheath 220 by a second length L2 longer than first length L1.

The body portion 15 is formed in a substantially columnar shape extending in the longitudinal direction A. The body portion 15 has an enlarged diameter portion 16, an engaging portion 17, and a connecting portion 18 connected to the connecting member 2. Note that the body portion 15 may be curved in the direction as the rod portion 14K.

The enlarged diameter portion 16 is provided on the distal end side A1 of the body portion 15 and connected to the rod-shaped portion 14K. The enlarged diameter portion 16 is formed in a planar shape in which the distal end side A1 is substantially perpendicular to the longitudinal direction A. The outer diameter of the enlarged diameter portion 16 is larger than the outer diameter of the rod-shaped portion 14K. That is, at the connecting portion between the body portion 15 and the rod-shaped portion 14K, the outer diameter of the body portion 15 is larger than the outer diameter of the rod-shaped portion 14K.

For example, the length of the puncturing portion 11 in the longitudinal direction A is approximately 5 mm. In addition, the length of the rod-shaped portion 14K in the longitudinal direction A (the length from the return portion 13 of the puncturing portion 11 to the enlarged diameter portion 16 of the body portion 15) is smaller than the spread width of a general clip, for example, about 10 mm to 20 mm long.

### [Operation of Medical Instrument 100K and Applicator 200K]

FIGS. 44 to 46 are diagrams explaining the operation of the medical instrument 100K and the applicator 200K. FIG. 45A is a side view of hook 270. FIG. 45B is a front view of hook 270 viewed from direction B in FIG. 45A. FIG. 45C is a cross-sectional view of hook 270 along line C-C in FIG. 45A. FIG. 45D is a cross-sectional view of hook 270 along line D-D in FIG. 45A.

As shown in FIG. 44, the connecting member 2 of the medical instrument 100K loaded in the applicator 200K is connected to the arrowhead hook portion 231 that passes through the sheath 220. The engaging portion 17 is pressed by the inner peripheral surface of the sheath 220 and is in a retracted state. The rod-shaped portion 14K of the medical device 100K is elastically deformed so as to follow the shape of the sheath 220.

As the slider 242 advances along the operation portion main body 241, the arrowhead hook portion 231 advances. As shown in FIG. 45A, the medical device 100K is advanced at least until the engagement portion 17 is out of the sheath 220.

By coming out of the sheath 220, the engaging portion 17 transitions from the retracted state to the protruding state, which is a basic posture. Since the engaging portion 17 in the projecting state engages with the distal tip 221 of the sheath 220, it is not pulled into the sheath 220. Therefore, the indwelling member 1K is not pulled into the sheath 220 after coming out of the sheath 220.

As shown in FIG. 45A, the rod-shaped portion 14K of the indwelling member 1K is released from elastic deformation as it protrudes from the sheath 220, and assumes a curved shape with respect to the longitudinal direction B. At this time, the puncturing portion 11 of the indwelling member 1K curves in a direction approaching the hook main body 271 of the hook 270. The puncturing portion 11 of the indwelling member 1K moves to the distal end side B1 of the hook distal end portion 272 of the hook 270. More specifically, the return portion 13 of the puncturing portion 11 moves to the distal end side B1 of the hook distal end portion 272. At this time, the puncturing portion 11 is inserted through the path 273 formed by the hook distal end portion 272.

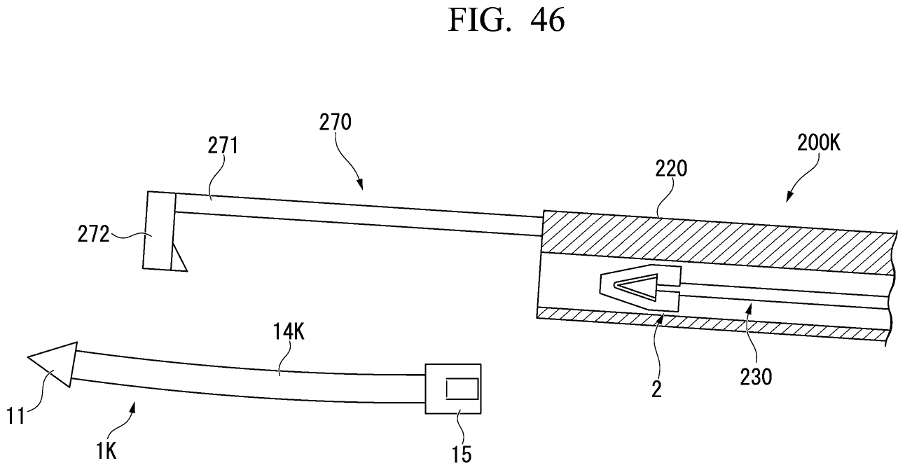

As shown in FIG. 46, the slider 242 retreats along the operation portion main body 241, whereby the arrowhead hook portion 231 retreats. The connecting member 2 connected to the arrowhead hook portion 231 pulls the indwelling member 1K. Since the engaging portion 17 in the projecting state engages with the distal tip 221 of the sheath 220, it is not pulled into the sheath 220. The distal end connecting portion 21 of the connecting member 2 is separated from the connecting portion 18 of the indwelling member 1K by, for example, a part of the distal end connecting portion 21 being broken. The connecting member 2 may be separated from the connecting portion 18 of the indwelling member 1K by, for example, elastic deformation or plastic deformation of part of the distal end connecting portion 21.

As shown in FIG. 46, in a state in which the connection between the wire 232 and the retention member 1K is released, the retention member 1K is released when the endoscope 300 and the sheath 220 are separated from the retention member 1K.

As described above, the seventh embodiment of the present disclosure has been described in detail with reference to the drawings, but the specific configuration is not limited to this embodiment, and design changes and the like are included within the scope of the present disclosure. Also, the constituent elements shown in the above-described embodiment and modifications shown below can be combined as appropriate.

What is claimed is:

1. An endoscopic suturing method, comprising:
penetrating an indwelling member into a first portion of a mucous membrane;
with the indwelling member penetrated into the first portion of the mucous membrane, moving the indwelling member toward a second portion of the mucous membrane while pushing the first portion of the mucous membrane and penetrating the indwelling member into the second portion of the mucous membrane; and
leaving the indwelling member in a body in a state in which the indwelling member penetrates the first portion of the mucous membrane and the second portion of the mucous membrane.

2. The endoscopic suturing method according to claim 1, wherein, in the state in which the indwelling member penetrates the first portion of the mucous membrane and the second portion of the mucous membrane, a gap is present between the first portion of the mucous membrane and the second portion of the mucous membrane.

3. The endoscopic suturing method according to claim 1, wherein, in a suturing direction, the first portion of the mucous membrane and the second portion of the mucous membrane face each other across a defective portion of the mucous membrane.

4. The endoscopic suturing method according to claim 3, wherein a width between the first portion of the mucous membrane and the second portion of the mucous membrane is a largest width in the suturing direction of the defective portion.

5. The endoscopic suturing method according to claim 1, further comprising:
pressing the indwelling member against the first portion of the mucous membrane before penetrating the indwelling member into the first portion of the mucous membrane.

6. The endoscopic suturing method according to claim 1, further comprising:
pressing the indwelling member against the second portion of the mucous membrane and before penetrating the indwelling member into the second portion of the mucous membrane.

7. The endoscopic suturing method according to claim 3, wherein the indwelling member is a first-type indwelling member, and
wherein the method further comprises:
penetrating a second first-type indwelling member through a third portion of the mucous membrane and a fourth portion of the mucous membrane, and
indwelling the second first-type indwelling member when a distance in the saturating direction between the third portion of the mucous membrane and the fourth portion of the mucous membrane is equal to or longer than a length that can be sutured by a second-type indwelling member,
wherein the third portion is a part of the first portion of the mucous membrane, and the fourth portion is a part of the second portion of the mucous membrane, and
wherein the third portion of the mucous membrane and the fourth portion of the mucous membrane face each other in the suturing direction.

8. The endoscopic suturing method according to claim 7, further comprising:
suturing the third portion of the mucous membrane and the fourth portion of the mucous membrane using the second type indwelling member.

9. The endoscopic suturing method according to claim 8, wherein the second-type indwelling member is a clip.

10. The endoscopic suturing method according to claim 1, further comprising:
before penetrating the indwelling member into the first portion of the mucous membrane, holding portions of the mucous membrane around the first portion of the mucous membrane.

11. The endoscopic suturing method according to claim 1, further comprising:
before penetrating the indwelling member into the second portion of the mucous membrane, holding portions of the mucous membrane around the second portion of the mucous membrane.

12. The endoscopic suturing method according to claim 1, further comprising:
after penetrating the indwelling member through the first portion of the mucous membrane, removing the indwelling member from the first portion, and penetrating the indwelling member through another portion of the mucous membrane.

13. The endoscopic suturing method according to claim 1, further comprising:
after penetrating the indwelling member into the second portion of the mucous membrane, pulling out the indwelling member from the second portion of the mucous membrane and penetrating the indwelling member through another portion of the mucous membrane.

14. The endoscopic suturing method according to claim 1, further comprising:
during penetrating the indwelling member into the first portion of the mucous membrane, regulating an amount of puncture of the indwelling member into the first portion of the mucous membrane.

15. The endoscopic suturing method according to claim 1, further comprising:
during penetrating the indwelling member into the second portion of the mucous membrane, regulating an amount of puncture of the indwelling member into the second portion of the mucous membrane.

16. The endoscopic suturing method according to claim 1, wherein, in the state in which the indwelling member penetrates the first portion of the mucous membrane and the second portion of the mucous membrane, the method further comprises:
removing at least a first part of the indwelling member while a second part of the indwelling member continues to penetrate the first portion of the mucous membrane and the second portion of the mucous membrane.

17. The endoscopic suturing method according to claim 1, wherein moving the indwelling member toward a second portion of the mucous membrane while pushing the first portion of the mucous membrane includes moving the first portion of the mucous membrane toward the second portion of the mucous membrane.
